(19) United States
(12) Patent Application Publication
(10) Pub. No.: US 2016/0151347 A1
(43) Pub. Date: Jun. 2, 2016

(54) PHARMACEUTICAL COMPOUNDS

(71) Applicant: ISIS Innovation LTD, Oxford (GB)

(72) Inventor: Rebecca Burton, Oxfordshire (GB)

(21) Appl. No.: 14/904,005

(22) PCT Filed: Jul. 10, 2014
(86) PCT No.: PCT/GB2014/052109

§ 371 Date: Jan. 8, 2016

(30) Foreign Application Priority Data
Jul. 10, 2013 (GB) 1312386.4

(51) Int. Cl. A61K 31/4706; A61K 45/06

(52) U.S. Cl. CPC A61K 31/4706; A61K 45/06

(57) ABSTRACT

The present invention provides hydroxychloroquine or a pharmaceutically acceptable salt thereof for use in the prevention and/or treatment of systolic heart failure, sincus tachycardia, cardiac syndrome X and/or essential hypertension.

This invention relates to new uses of hydroxychloroquine, specifically, the use of hydroxychloroquine in the prevention or treatment of systolic heart failure, diastolic heart failure, sinus tachycardia, cardiac syndrome X and/or essential hypertension.

Cardiovascular disease is a collective term given to diseases that affect the heart. It is known in the art that a high heart rate can increase the risk of cardiovascular disease and that the reduction of heart rate can treat such cardiovascular diseases and also be of general benefit to the health of a patient, as discussed in Progress in Cardiovascular Diseases 52 (2009) 46-60, Elevated Heart Rate in Cardiovascular Diseases: A Target for Treatment?, Paolo Palatini.

Two widely used groups of drugs that directly lower heart-rate are certain (L-type) calcium channel antagonists and beta-adrenoceptor antagonists (beta blockers). Although both are widely used therapeutically for the treatment of cardiovascular diseases, neither drug is cardio-selective in its actions.

Beta blockers are currently used to treat angina, arrhythmias, heart failure, hypertension and myocardial infarction. Beta blockers act to alter both heart rate and blood pressure. Examples of beta blockers are propranolol, atenolol, bisoprolol, metoprolol, esmolol and sotalol. However, there are various drawbacks of treatment with beta blockers, including symptoms associated with the central nervous system reaction and symptoms associated with beta-2 adrenergic antagonistic activity. Such drawbacks include bradycardia, heart block, cardiac failure, bronchospasm, cold extremities and claudication, Raynaud's phenomenon, worsening of asthma symptoms, nausea, anorexia, vomiting, diarrrhoea, fatigue, muscle cramps, weakness, dizziness, lassitude, ataxia, anxiety, confusion, hallucinations, vivid dreams and sleep disturbance. Beta-blockers are contraindicated in asthma and COPD and shouldn't be used unless there is no alternative, in which case a “cardiac-specific” version must be started under specialist supervision (NICE guidelines). Sudden withdrawal of beta blockers may result in cardiac arrhythmias or an exacerbation of symptoms of ischemic heart disease, sometimes with the occurrence of myocardial infarction. In addition, because of their short half-life (3 to 10 hours), beta blockers must be taken daily to be effective. Because a cardiovascular disease such as hypertension is a life-long disorder, longer-lasting treatment without side effects would be desirable.

Calcium channel blockers (calcium channel antagonists) are a group of drugs that affect the way calcium enters into certain muscle cells. Calcium channel blockers are used to treat various conditions including blood pressure, angina, Raynaud's phenomenon, some arrhythmias, circulatory disorders, hypertension, left ventricular dysfunction (LVD) and myocardial infarction. Examples of calcium channel blockers include amlodipine, diltiazem, felodipine, isradipine, lacidipine, lercanidipine, nicardipine, nifedipine, nisoldipine and verapamil. A calcium channel blocker can be used alone but often, it is used as a combination therapy with another drug (such as angiotensin converting enzyme inhibitors) to treat high blood pressure, particularly when one drug has not worked well when administered alone. Rate-limiting calcium blockers act more on the heart to lower heart rate. The possible common side effects associated with calcium channel blockers include the development of flushing and headaches, mild ankle swelling and constipation. Other side effects include nausea, palpitations, malaise, fatigue, dizziness and rashes. Other considerations include cases where a patient suddenly stops taking a calcium channel blocker and experiences a rebound flare of angina.

Ivabradine (Procoralan, S 16257, S 162572, S-15544, S-16260) is a sinus node I_{f} current inhibitor and a sino-atrial modulator with bradycardic activity. It acts to alter heart rate by selectively inhibiting inward Na+/K+ cardiac current through the hyperpolarisation-activated “funny current” channels, slowing the onset of the next action potential and therefore the next heart beat, but has little effect on blood pressure. It is used to treat cardiovascular disease and known to treat systolic heart failure, diastolic heart failure, sinus tachycardia, cardiac syndrome X and/or pulmonary hypertension. This is the only selective heart rate reducing agent currently available for clinical use.

There is therefore a need in the art for alternative substances for preventing or treating systolic heart failure, diastolic heart failure, sinus tachycardia, cardiac syndrome X and/or essential hypertension.

Accordingly, the present invention provides hydroxychloroquine (HCQ) or a pharmaceutically acceptable salt thereof for use in the prevention and/or treatment of systolic heart failure, diastolic heart failure, sinus tachycardia, cardiac syndrome X (CSX) and/or essential hypertension.

It has been surprisingly found that hydroxychloroquine is a bradycardic agent that acts by directly inhibiting the I_{f} current and is hence a sinus node I_{f} inhibitor with bradycardic activity. The inventors have shown that hydroxychloroquine lowers heart rate and owing to its mode of action of blocking the I_{f} current, can be used to prevent and/or treat systolic heart failure, diastolic heart failure, sinus tachycardia, cardiac syndrome X and/or essential hypertension.

The present invention will now be described with reference to the accompanying drawings in which:

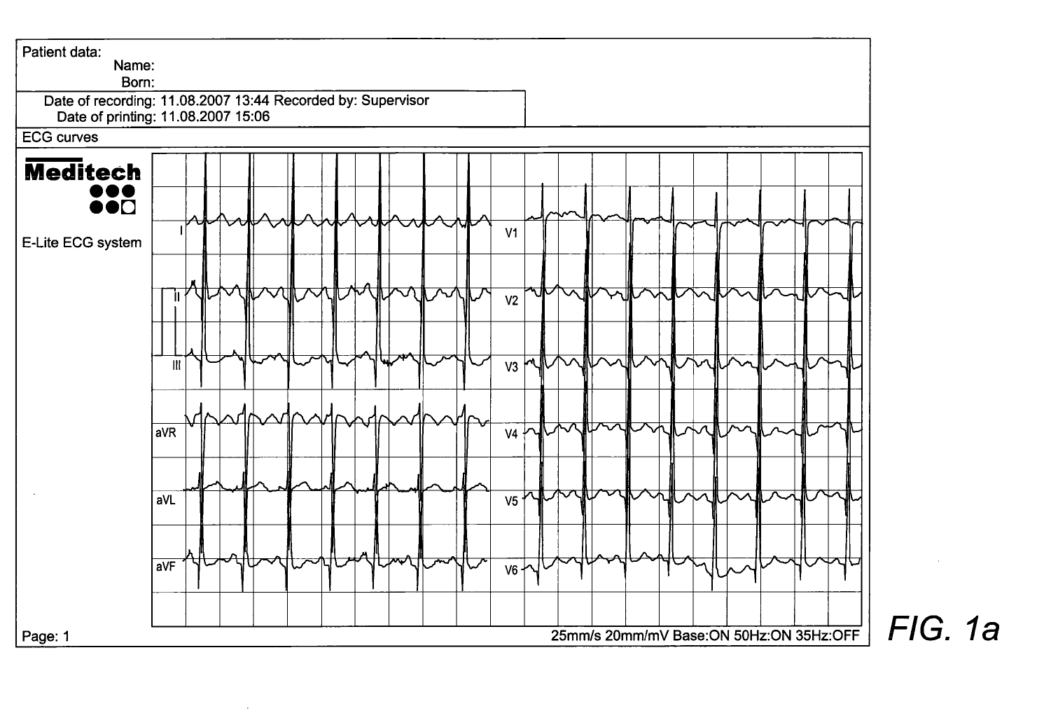

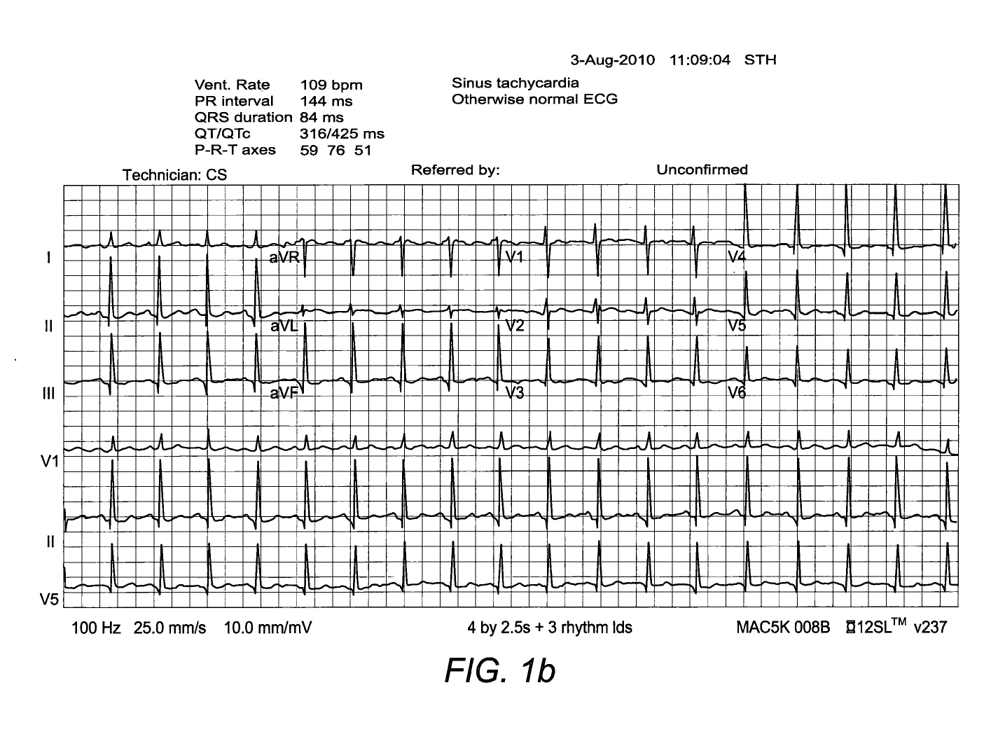

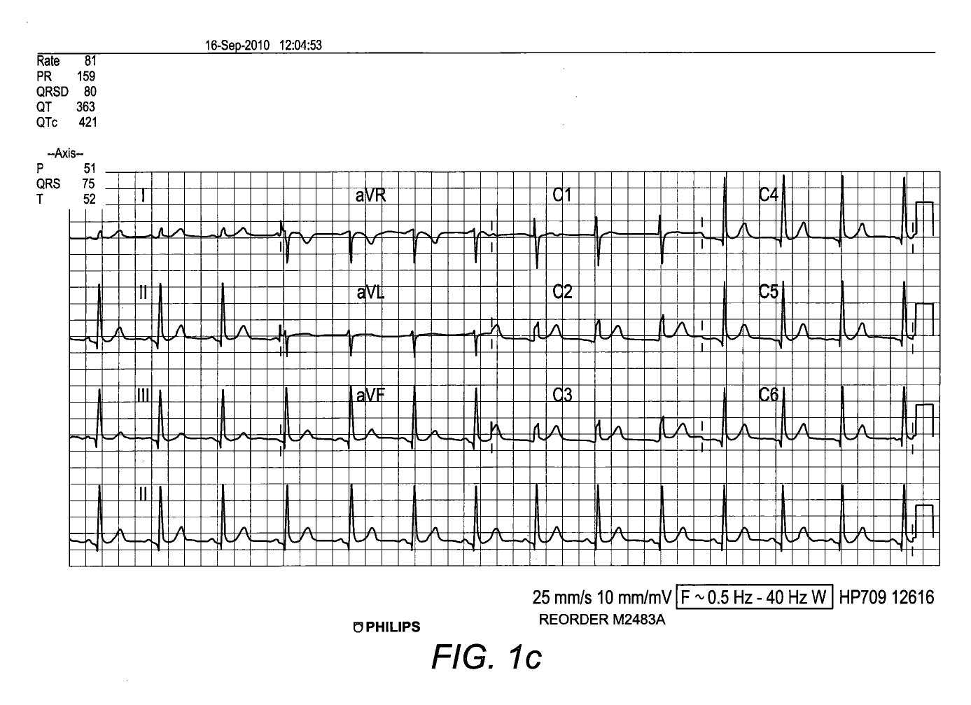

FIG. 1a shows an electrocardiogram of a human systemic lupus erythematosus patient with idiopathic tachycardia as detailed in Example 1;

FIG. 1b shows an electrocardiogram for a human systemic lupus erythematosus patient with idiopathic sinus tachycardia in Example 1 after a reduction in dose of hydroxychloroquine;

FIG. 1c shows an electrocardiogram for a human patient of Example 1 showing normal sinus rhythm upon treatment with low dose hydroxychloroquine (200 mg daily);

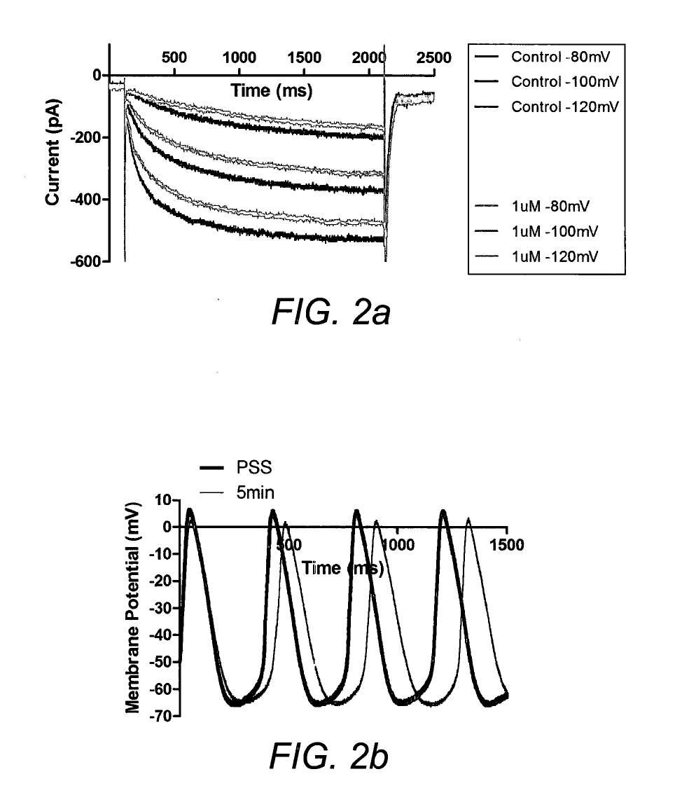

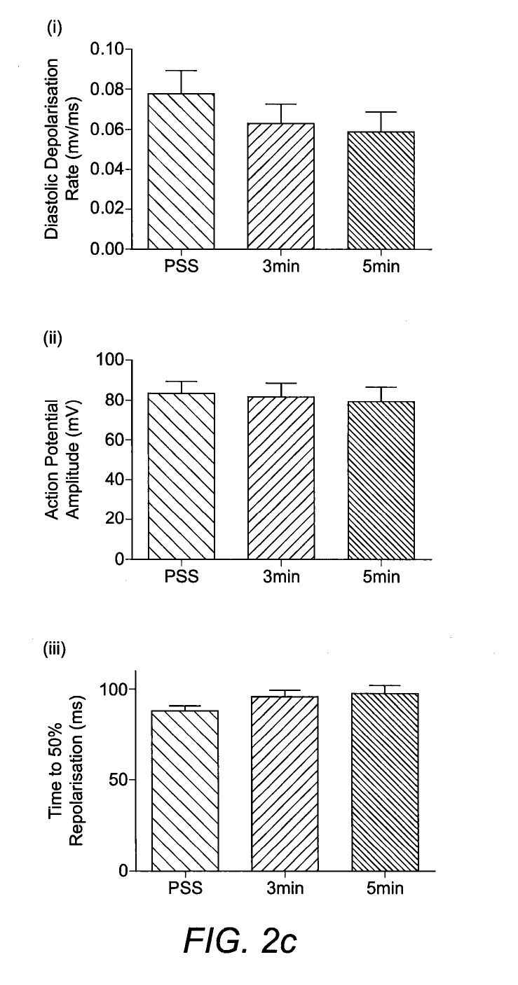

FIG. 2a shows a representative trace detailing the bradycardic effects of hydroxychloroquine on I_{f} in a single isolated sinoatrial node cell using the perforated patch clamp technique which shows that 1 μM of hydroxychloroquine causes a significant reduction in I_{f} current;

FIG. 2b shows an action potential trace control and drug trace showing slowing of the action potential over a 5 minute period of hydroxychloroquine treatment;

FIG. 2c shows action potential and diastolic depolarization rate in isolated guinea-pig sino-atrial node cells;

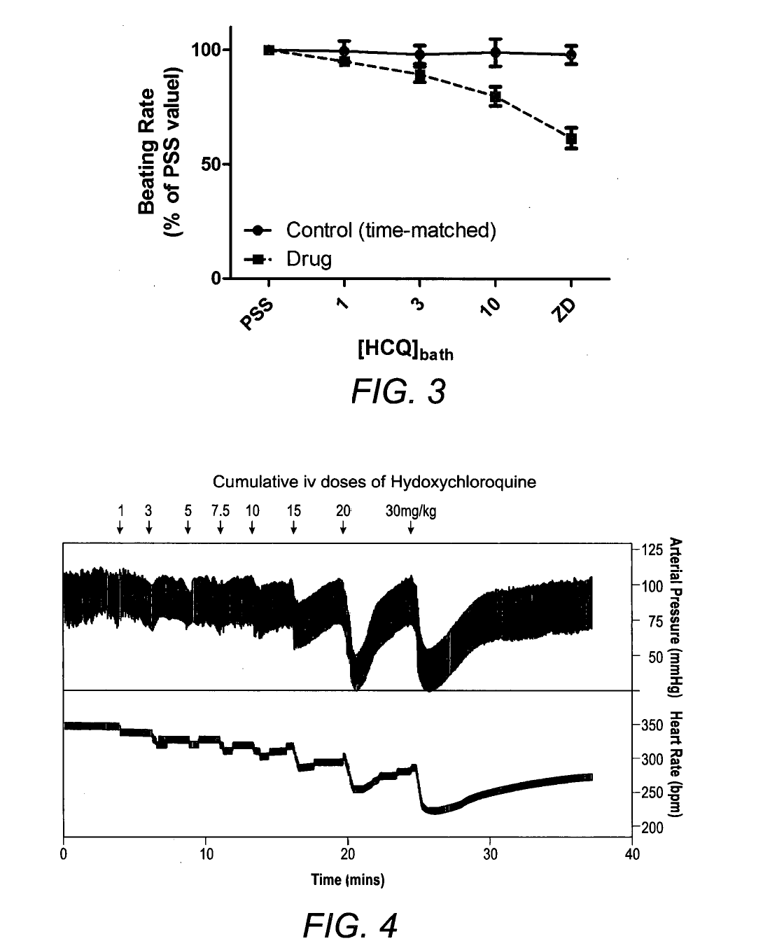

FIG. 3 shows a graph detailing the effects of hydroxychloroquine and ZD7288 on mouse atrial tissue preparations with intact sino-atrial node which shows percentage change in beating during a 30 minute exposure to 1 μM, 3 μM, 10 μM and 1 μM ZD7288 (in the presence of 10 μM HCQ) respectively;

FIG. 4 shows a trace from an in vivo anaesthetised, adult SD rat (male) showing blood pressure and heart rate responses (measured directly via a left common carotid arterial cannula) to cumulative intravenous doses of hydroxychloroquine;

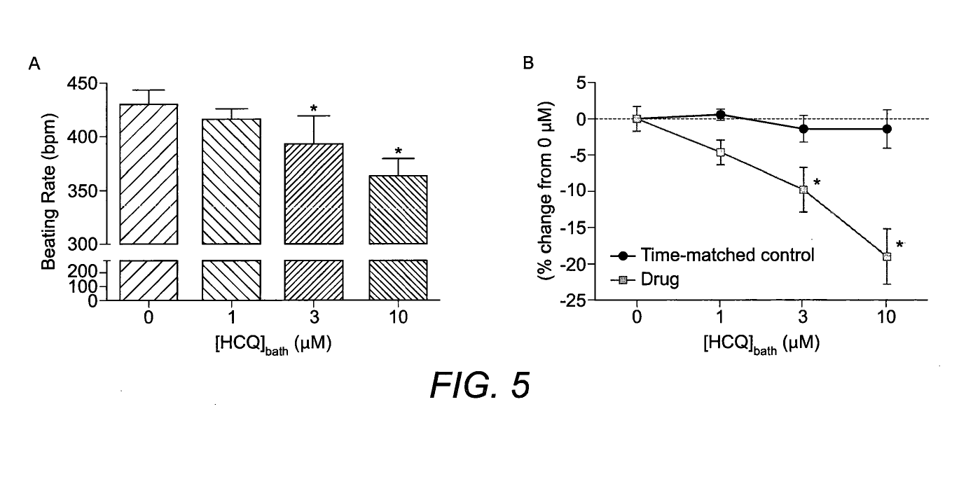

FIG. 5 shows the dose-dependent effect of hydroxychloroquine on spontaneous beating rate in mouse atrial preparations where n=7 for all concentrations;

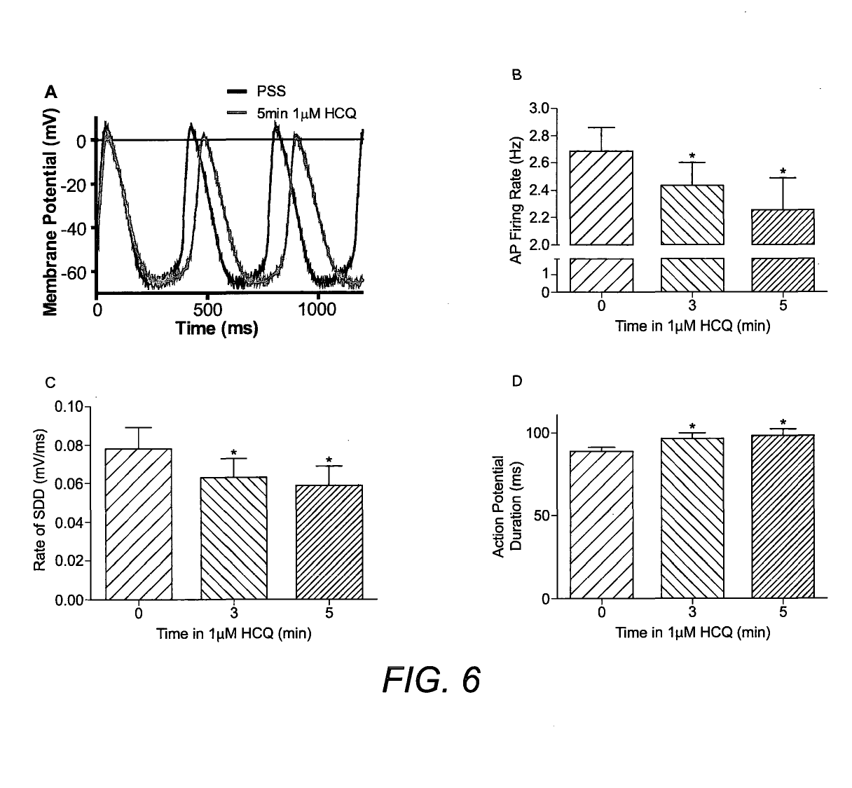

FIG. 6 shows electrophysiology single cell studies on sinoatrial nodal cells investigating the effect of hydroxychloroquine on the action potential where n=6;

FIG. 7 shows electrophysiology single cell studies on sinoatrial nodal cells investigating the current-voltage relations in the presence of 3 μM HCQ and wash out;

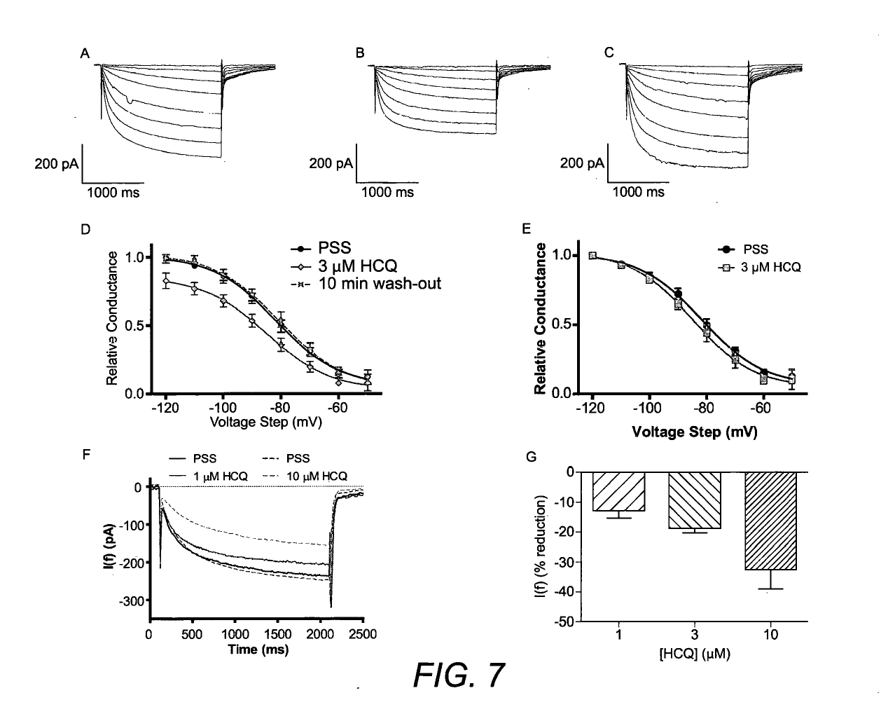

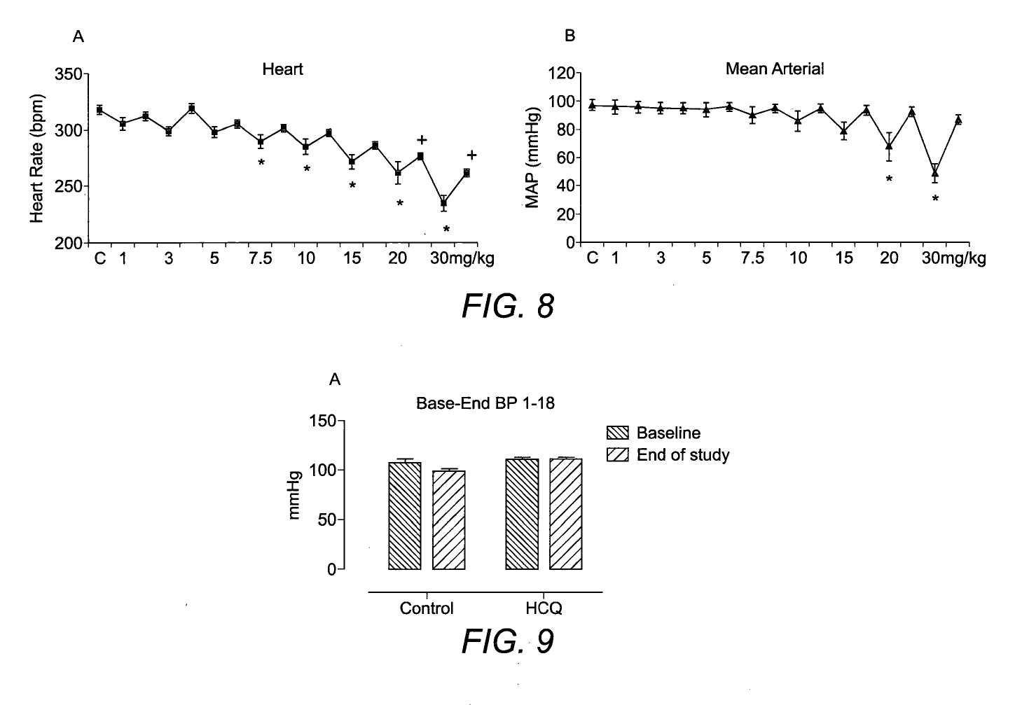

FIG. 8 shows acute in-vivo experiments of anaesthetised (2% isofluorane), adult SD rat (male), of heart rate and blood pressure responses (measured directly via left carotid cannula) to cumulative intravenous doses of vehicle; and

FIG. 9 shows the effects of oral hydroxychloroquine on blood pressure by tail cluff plethysmography (A), where n=9/group and dose 100 mg/kg in drinking water for 2 weeks.

Hydroxychloroquine (chemical name: 2-[{4-[(7-chloroquinolin-4-yl)amino]pentyl}(ethyl)amino]ethanol) has been known since the early 1950s (U.S. Pat. No. 2,546,658). Initially developed as an antimalarial drug and sold as the sulfate salt by Sanofi Aventis under the trade name Plaquenil®, hydroxychloroquine sulfate is also used for the treatment of rheumatoid arthritis and inflammatory skin diseases, including systemic lupus erythematosus.

The chemical structure of hydroxychloroquine is as shown below.

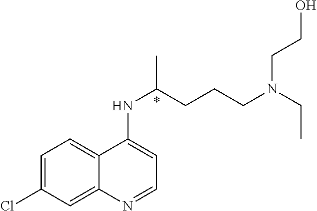 [SMILES: CCN(CCO)CCCC(C)Nc1ccnc2cc(Cl)ccc12]

The compound has a chiral centre at the carbon atom that is identified with an asterisk and hence can exist in two enantiomeric forms, (R)-(−)-2-[{4-[(7-chloroquinolin-4-yl)amino]pentyl}(ethyl)amino]ethanol) [hereinafter (R)-(−)-hydroxychloroquine] and (S)-(+)-2-[{4-[(7-chloroquinolin-4-yl)amino]pentyl}(ethyDamino]ethanol) [hereinafter (S)-(+)-hydroxychloroquine].

Hydroxychloroquine can therefore exist as a racemate consisting of a 1:1 mixture of two enantiomers, substantially the single (R)-(−)-hydroxychloroquine or substantially the single (S)-(+)-hydroxychloroquine. Commercially available hydroxychloroquine is a racemic mixture of the two enantiomers.

Methods for the preparation of hydroxychloroquine racemate or isomers are known in the art. For example, hydroxychloroquine can be prepared according to U.S. Pat. No. 2,546,658 and U.S. Pat. No. 5,314,894.

The data presented herein has shown that hydroxychloroquine acts to reduce cardiac rate by directly inhibiting the I_{f} current and is hence a sinus node I_{f} inhibitor with bradycardic activity. Given that heart rate is one of the major determinants of prognosis in patients with cardiac dysfunction, and following the known example of ivabradine, this allows for the prevention and/or treatment of systolic heart failure, diastolic heart failure, sinus tachycardia, cardiac syndrome X and/or essential hypertension.

It is shown herein that hydroxychloroquine or a pharmaceutically acceptable salt thereof may be used to prevent and/or treat systolic heart failure, diastolic heart failure, sinus tachycardia, cardiac syndrome X and/or essential hypertension. Any reference herein to hydroxychloroquine also includes reference to its pharmaceutically acceptable salts.

Studies carried out in vivo and ex vivo show its powerful specific activity of reducing cardiac frequency and bringing about a reduction in heart rate via HCN (I_{f}) specific agents. The studies performed confirm that at the concentrations used, the activity of HCQ is directly on the sinoatrial node. This effect on cardiac frequency is not accompanied by a detrimental effect on the arterial pressure or sino-atrial node action potential.

As used herein, the terms “prevent” and “treat” encompass the prevention of the development of a disease or a symptom in a patient who may have a predisposition of the disease or the symptom but has not yet been diagnosed to have the disease or the symptom; the inhibition of the symptoms of a disease, namely, inhibition or retardation of the progression thereof; and the alleviation of the symptoms of a disease, namely, regression of the disease or the symptoms, or inversion of the progression of the symptoms.

Systolic heart failure, diastolic heart failure, sinus tachycardia (sinus node re-entrant tachycardia), cardiac syndrome X and essential hypertension are well known diseases in the art and a detailed description is therefore not required.

Heart failure (or chronic heart failure) occurs when the heart is unable to provide sufficient pump action or cardiac output to maintain the blood flow needs of the body. Systolic heart failure (SHF) is heart failure caused by systolic dysfunction, wherein the heart fills adequately but does not empty to a normal extent, leading to a decreased ejection fraction. Systolic dysfunction is from impaired contractile or pump action. Diastolic heart failure (DHF) is heart failure caused by diastolic dysfunction, wherein inadequate diastolic filling of the heart occurs. Diastolic dysfunction is from impaired ventricular relaxation, compliance or filling.

Sinus tachycardia is a heart rhythm with elevated rate of impulses originating from the sinoatrial node. Sinus tachycardia is defined by a heart rate of greater than 100 bpm. Sinus tachycardia includes sinus node re-entrant tachycardia.

Patients with cardiac syndrome X typically experience angina pectoris but have no flow limiting lesions in their main epicardial coronary arteries when explored with angiography. Although prognosis is good regarding survival, patients with CSX have an impaired quality of life.

The causes of human essential hypertension remain unknown. Progress towards defining the genetic basis of susceptibility to hypertension has been slow due to the complexity of arterial blood pressure. It is established that in many patients, sympathetic nerve activity (which increase stimulation on the heart and increase heart rate), increases proportionately as hypertension develops and this may be a causative factor, but it is unclear what triggers heightened sympathetic traffic.

Particularly preferred pharmaceutically acceptable salts of hydroxychloroquine for use in the present invention are acid addition salts. Preferred acid addition salts include those formed with sulfuric acid. Particularly preferred is hydroxychloroquine sulfate, for example Plaquenil®.

Acid addition salts can be prepared by the methods described herein or conventional chemical methods such as the methods described in Pharmaceutical Salts: Properties, Selection, and Use, P. Heinrich Stahl (Editor), Camille G. Wermuth (Editor), ISBN: 3-90639-026-8, Hardcover, August 2002.

Generally, such salts can be prepared by reacting the free base form of the compound with the appropriate base or acid in water or in an organic solvent, or in a mixture of the two; generally, nonaqueous media such as ether, ethyl acetate, ethanol, isopropanol, or acetonitrile are used.

Hydroxychloroquine may form N-oxides. Particular examples of N-oxides are the N-oxides of a tertiary amine or a nitrogen atom of a nitrogen-containing heterocycle.

N-oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Hydroxychloroquine contains a chiral centre and can exist in the form of two optical isomers ((R)-(−)-hydroxychloroquine and (S)-(+)-hydroxychloroquine). References to hydroxychloroquine include all optical isomeric forms thereof, either as individual optical isomers, or mixtures (e.g. racemic mixtures), unless the context requires otherwise.

One enantiomer of hydroxychloroquine in a pair of enantiomers may exhibit advantages over the other enantiomer, for example, in terms of biological activity. Thus, in certain circumstances, it may be desirable to use as a therapeutic agent only one of a pair of enantiomers. Accordingly, the invention provides compositions containing hydroxychloroquine having one chiral centre, wherein at least 90% (e.g. at least 95%, 98% or 99%) of the compound of hydroxychloroquine is present as a single optical isomer (e.g. (R)-(−)-hydroxychloroquine or (S)-(+)-hydroxychloroquine). In one general embodiment, 99% or more (e.g. substantially all) of the total amount of hydroxychloroquine may be present as a single optical isomer.

Hydroxychloroquine is typically administered in the form of a pharmaceutical composition.

The pharmaceutical composition may be in any suitable form of administration, e.g. oral or parenteral. Where the composition is intended for parenteral administration, it can be formulated for intravenous, intramuscular, intraperitoneal, subcutaneous administration or for direct delivery into a target organ or tissue by injection, infusion or other means of delivery such as suppositories.

Preferably, the pharmaceutical dosage form is suitable for oral administration, which includes tablets, capsules, caplets, pills, lozenges, syrups, solutions, sprays, powders, granules, elixirs and suspensions, sublingual tablets, sprays, wafers or patches and buccal patches. Preferably, the pharmaceutical dosage form is that of a tablet, most preferably a coated tablet.

Pharmaceutical compositions containing hydroxychloroquine can be formulated in accordance with known techniques, see for example, Remington's Pharmaceutical Sciences, Mack Publishing Company, Easton, Pa., USA, 22nd Edition.

The hydroxychloroquine of the invention will generally be presented in unit dosage form and, as such, will typically contain sufficient compound to provide a desired level of biological activity. For example, a formulation intended for oral administration may contain from 1 milligrams to 1000 milligrams of active ingredient, more usually from 200 milligrams to 800 milligrams, for example, 300 milligrams, 400 milligrams, 500 milligrams, 600 milligrams or 700 milligrams.

The active compound will be administered to a patient in need thereof (for example a human or animal patient) in an amount sufficient to achieve the desired therapeutic effect.

The patient in need of such administration is a patient suffering from or exhibiting, or at risk of suffering from or exhibiting, systolic heart failure, diastolic heart failure, sinus tachycardia, cardiac syndrome X and/or essential hypertension.

The desired therapeutic effect may be the prevention, alleviation or reduction of systolic heart failure, diastolic heart failure, sinus tachycardia, cardiac syndrome X and/or essential hypertension or one or more symptoms thereof. Such symptoms are well known to the skilled person (e.g. a skilled physician) who will be able to judge through clinical evaluation and testing in a conventional manner whether or not the administration of a compound of the invention has resulted in a change in the symptoms exhibited by the patient.

The compounds will typically be administered in amounts that are therapeutically or prophylactically useful and which generally are non-toxic. However, in certain situations, the benefits of administering hydroxychloroquine may outweigh the disadvantages of any toxic effects or side effects, in which case it may be considered desirable to administer compounds in amounts that are associated with a degree of toxicity.

A typical daily dose of the compound for a human patient may be up to 1000 mg per day, preferably from 50 to 800 milligrams per day, more preferably from 100 to 800 milligrams per day, for example 200 mg, 300 mg, 400 mg, 500 mg, 600 mg or 700 mg per day. Most preferred is 1 to 400 milligrams per day, more preferably from 1 to 200 mg per day and 200 to 400 mg per day. Typically, this may be administered in the form of one to four 200 milligrams tablet per day.

Doses for other mammals and for children may vary. Preferably the daily dose of the compound will be in the range of from 0.01 milligrams to 20 milligrams per kilogram of body weight, more usually from 0.025 milligrams to 10 milligrams per kilogram of body weight, for example up to 7.5 milligrams per kilogram of body weight, and more typically 0.10 milligrams to 7.5 milligrams per kilogram of body weight, although higher or lower doses may be administered where required.

Ultimately, however, the quantity of compound administered will be commensurate with the nature of the disease or physiological condition being prevented or treated and the therapeutic benefits and the presence or absence of side effects produced by a given dosage regimen, and will be at the discretion of the physician.

In another embodiment, the present invention provides hydroxychloroquine (HCQ) or a pharmaceutically acceptable salt thereof for use in the prevention and/or treatment of systolic heart failure, diastolic heart failure, sinus tachycardia and/or cardiac syndrome X (CSX).

The invention also provides the combination of hydroxychloroquine or a pharmaceutically acceptable salt thereof and an ACE inhibitor, an angiotensin II receptor blocker, a diuretic, an aldosterone antagonist, a calcium channel antagonist, a beta blocker and/or a sinus node I_{f} inhibitor for use in the prevention and/or treatment of systolic heart failure, diastolic heart failure, sinus tachycardia, cardiac syndrome X and/or essential hypertension.

The description hereinabove in relation to the use of hydroxychloroquine applies equally in relation to the combination.

The components of the combination may be administered sequentially, separately or simultaneously and this may be determined by the physician based on his knowledge and the patient in question. Preferably, hydroxychloroquine is to be administered sequentially, separately or simultaneously with an ACE inhibitor, an angiotensin II receptor blocker, a diuretic, an aldosterone antagonist, a calcium channel antagonist, a beta blocker and/or a sinus node I_{f} inhibitor.

As used herein, the term “combination”, as applied to two or more compounds, may define material in which the two or more compounds are associated. The terms “combined” and “combining” in this context are to be interpreted accordingly.

The association of the two or more compounds in a combination may be physical or non-physical. Examples of physically associated combined compounds include:
compositions (e.g. unitary formulations) comprising the two or more compounds in admixture (for example within the same unit dose);
compositions comprising material in which the two or more compounds are chemically/physicochemically linked (for example by crosslinking, molecular agglomeration or binding to a common vehicle moiety);
compositions comprising material in which the two or more compounds are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds are co-packaged or co-presented (e.g. as part of an array of unit doses);

Examples of non-physically associated combined compounds include:
material (e.g. a non-unitary formulation) comprising at least one of the two or more compounds together with instructions for the extemporaneous association of the at least one compound to form a physical association of the two or more compounds;
material (e.g. a non-unitary formulation) comprising at least one of the two or more compounds together with instructions for combination therapy with the two or more compounds;
material comprising at least one of the two or more compounds together with instructions for administration to a patient population in which the other(s) of the two or more compounds have been (or are being) administered;
material comprising at least one of the two or more compounds in an amount or in a form which is specifically adapted for use in combination with the other(s) of the two or more compounds.

As used herein, the term “combination therapy” is intended to define therapies which comprise the use of a combination of two or more compounds (as defined above). Thus, references to “combination therapy”, “combinations” and the use of compounds “in combination” in this application may refer to compounds that are administered as part of the same overall treatment regimen. As such, the posology of each of the two or more compounds may differ: each may be administered at the same time or at different times. It will therefore be appreciated that the compounds of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds in a combination therapy may also differ with respect to the route of administration.

The invention also provides a pharmaceutical kit, a pharmaceutical pack and a patient pack, comprising hydroxychloroquine alone or in combination with an ACE inhibitor, an angiotensin II receptor blocker, a diuretic, an aldosterone antagonist, calcium channel antagonist, a beta blocker and/or a sinus node I_{f} inhibitor with instructions for use in the prevention and/or treatment of systolic heart failure, diastolic heart failure, sinus tachycardia, cardiac syndrome X and/or essential hypertension, as discussed above.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds, the individual compounds may be unitary or non-unitary formulations. The unit dose(s) may be contained within a blister pack. The pharmaceutical kit further comprises instructions for use in the prevention and/or treatment of systolic heart failure, diastolic heart failure, sinus tachycardia, cardiac syndrome X and/or essential hypertension.

As used herein, the term “pharmaceutical pack” defines an array of one or more unit doses of a pharmaceutical composition, optionally contained within common outer packaging. In pharmaceutical packs comprising a combination of two or more compounds, the individual compounds may be unitary or non-unitary formulations. The unit dose(s) may be contained within a blister pack. The pharmaceutical pack further comprises instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The combinations of the invention may produce a therapeutically efficacious effect relative to the therapeutic effect of the individual compounds when administered separately.

The invention also provides a method for preventing or treating systolic heart failure, diastolic heart failure, sinus tachycardia, cardiac syndrome X and/or essential hypertension, wherein the method comprises administering to the mammal a therapeutically effective amount of hydroxychloroquine or a pharmaceutical acceptable salt thereof.

The description hereinabove in relation to the uses applies equally in relation to the method, as it does to the use of hydroxychloroquine alone or in combination.

### EXAMPLES

The following non-limiting examples illustrate the properties of hydroxychloroquine in accordance with the invention.

The experimental investigations in Examples 2 to 8 conform to the UK Home Office guidance on the Operation of Animals (Scientific Procedures) Act of 1986.

The heart rate of a mammal is defined as the number of heart beats per unit of time, usually heart beats per minute (bpm). The heart rate or number of heart beats per unit of time can be determined using methods known in the art. Such a method may involve the simple measurement of a mammal's pulse. Alternatively, heart rate may be measured through the use of an electrocardiograph (ECG), which is readily available to those skilled in the art. Such a method involves counting the number of QRS complexes in the ECG.

### Example 1

The patient was a 26 year old, female with no previous history of cardiac related disease.

In 2007, the patient experienced sinus tachycardia and had a heart rate of around 118 bpm (at rest), see FIG. 1a. The patient also experienced angina. After a coronary angiogram (which reassured that the patient had unblocked arteries), echocardiogram (which reassured that the patient had no structural problems with the heart) and cardiac stress test (heart rate between 175-190 bpm), following repeated tachycardic episodes, the patient was treated with atenolol (beta blocker) and a glyceryl trinitrate spray for angina. This treatment controlled the heart rate and reduced the heart rate to an acceptable rate of 70-80 bpm. Furthermore, the glyceryl trinitrate spray was used whenever angina symptoms were experienced. The patient experienced tiredness associated with the onset of use of atenolol, low blood pressure and increased effects of Raynaud's syndrome.

The patient attempted to stop treatment of Atenolol on several occasions. In Mar. 5 2008, the Atenolol was withdrawn for a short period in hospital during major surgery, due to low blood pressure. The patient experienced angina and documented arrhythmias. Consequently, the patient immediately resumed treatment with Atenolol and this alleviated the cardiac symptoms.

In June 2009, the patient was diagnosed with systemic lupus erythematosus. Consequently, she was treated with 200 mg to 400 mg of Plaquenil (hydroxychloroquine) per day to treat the systemic lupus erythematosus.

After a few months, the patient began to feel better and under the physician's guidance, the patient was gradually weaned off Atenolol. Within two weeks, Atenolol treatment was completely ceased and the patient experienced no tachyarrhythmias or angina. Subsequently, she has experienced no cardiac related problems and her heart rate remains at 70-80 bpm. Furthermore, the patient has a normal, healthy blood pressure.

In July 2010, the dosage of hydroxychloroquine that the patient was receiving was halved to 100 mg per day. In August 2010, the patient experienced tachyarrhythmias. On Aug. 3, 2010 the patient had an electrocardiogram due to a fast heart rate and angina over 4-5 days, see FIG. 1b. The recorded heart rate was 109 bpm. On Aug. 4, 2010 the patient resumed the treatment of 200 mg for systemic lupus erythematosus of hydroxychloroquine daily. On Sep. 16, 2010 the patient's heart rate was measured and was reduced to around 80-88 bpm, see FIG. 1c. The increase in dosage of hydroxychloroquine would appear to have contributed to this effect on reducing and stabilizing the heart rate. From August 2010 until December 2010, the patient has been running a stable heart rate between 70-85 bpm.

### Example 2

This example studies the effects of hydroxychloroquine on ion channels in isolated sinoatrial nodal cells using patch clamp electrophysiological techniques. The method and results are as follows:

### Guinea Pig Sino-Atrial Node Cell Isolation

Male guinea pigs (350-500 g) were sacrificed by concussion followed by cervical dislocation. The heart was rapidly excised and encouraged to beat several times in heparin-containing zero-Ca ²⁺ modified Tyrode solution to prevent clotting (10 U/mL). The heart was mounted via the aorta on a constant pressure Langendorff system for retrograde perfusion, with solutions maintained at body temperature. After 2 minutes initial wash with zero Ca ²⁺ solution (in mM: NaCl 136, KCI 5.4, NaHCO₃ 12, Na⁺ pyruvate 1, NaH₂PO₄ 1, MgCl₂ 1, EGTA 0.04, glucose 5; gassed with 95% O₂/5% CO₂ to maintain a pH of 7.4), perfusion was switched to re-circulation with collagenase solution (same as zero Ca²⁺ but with 27 mg collagenase/50 mL—type II Worthington, Worthington Biochemical Corp., Lakewood, N.J., USA—0.1 mM CaCl₂ and no EGTA).

Following 25 minutes enzymatic digestion, the right atrium, vena-cavae and pulmonary artery were dissected away from the ventricles. The right atrium was pinned in a Sylgard-coated bath, opened by anterior incision, and the SA node identified from anatomical features and tissue appearance. Under a binocular microscope, the SA node appears as a translucent, smooth, white structure in contrast to the thicker, pinker surrounding atrial tissue. The SA node was dissected into thin strips 2×5 mm) and stored in a high potassium medium (in mM: KCI 70, MgCl₂ 5, K⁺ glutamine 5, taurine 20, EGTA 0.04, succinic acid 5, KH₂PO₄ 20, HEPES 5, glucose 10; pH to 7.2 with KOH). Isolation of single SA node myocytes was carried out by gentle trituration.

For all single cell experiments, cells were transferred to a glass coverslip and superfused with oxygenated PSS at 34 ±1° C. using a gravity-fed system running at 2-3 mL/min. HCQ was applied via superfusion, with an electronic switching mechanism resulting in full solution exchange over a period of less than 20 seconds.

### Single Cell Electrophysiology Recordings

Perforated patch clamp recordings were carried out using electrodes pulled from filamented borosilicate glass capillary tubing (GC100 F, Harvard Apparatus Ltd, Kent) using a two-stage vertical puller (Narishige, Japan). Electrode resistances were in the range of 2.8-6.4 MOhm when filled with patch pipette solution containing (in mM): K⁺-aspartate 110, KCl 10, NaCl 5, MgCl₂ 5.2, HEPES 5, K₂ATP 5, pH to 7.2 with KOH. Patch solution also contained amphotericin (250 μg mL⁻¹) to achieve perforation.

Micropipettes were mounted in a Perspex holder containing an Ag/AgCl wire and connected to a CV203BU headstage. All recordings were carried out using an AxoPatch 200B microelectrode system, digitised with a Digidata 1200 A-D converter, and recorded using PCIamp7 software at a sampling rate of 2 kHz (All products in this paragraph, Axon Instruments).

Micropipettes were controlled using an electronic micromanipulator (Burleigh, USA). After positioning close to the cell of interest, the tip potential was manually compensated. Gigaseals were formed by manual suction and the cell left for up to 15 minutes to allow patch perforation and good electrical access using amphotericin. Cells were used in either current clamp mode for the recording of action potentials or voltage clamp mode for the recording of I_{f} currents.

### Action Potential Recordings

Action potentials were recorded from single guinea pig SA node cells under current clamp conditions using the AxoPatch200B microelectrode system and rate measured in Hz using spectral analysis.

### I_{f} Current Recordings

Cell voltage was clamped at −40 mV in the V-clamp configuration. Hyperpolarising pulses of 2 seconds in length were applied at 20 seconds intervals to measure I_{f} current. Current-voltage relations were taken at 0 minutes, 3 minutes and 5 minutes exposure to HCQ by application of successive steps to −80, −100 and −120 mV. Intervening voltage pulses were applied to −100 mV only. Maximum current was measured as the difference between maximum current during the voltage pulse and minimum current at the start of the voltage pulse.

### Results

Experiments using patch clamp techniques show the bradycardic effects of hydroxychloroquine on single isolated SA node, see FIG. 2a and b. At the lowest tested dose (1 μM) there is a significant reduction in I_{f} current after 5 min exposure to HCQ. This amounted to a reduction of 20+/−4% at the −80 mV voltage step and 12+/−4% at the −100 mV step (both data p<0.05, n=7, paired t-test).

Action potential measurements, see FIGS. 2b and 2c, carried out on isolated sino-atrial node cells using the current clamp method show a significant slowing in the rate of spontaneous action potential generation, 17+1-6% reduction after 5 min superfusion with 1 μM HCQ. This is accompanied by a 25+/−3% reduction in the slope of spontaneous diastolic depolarization, the portion of the action potential during which I_{f} is active, and an 11+/-3% lengthening in the time taken to reach 50% repolarisation.

### Example 3

This example studies the chronotropic effects of hydroxychloroquine on isolated mouse atrial preparation. The frequency of the spontaneous contractile activity is measured using tension transducer techniques. The method and results are as follows:

### Mouse Atrial Preparations

Male CD-1 mice (7-9 weeks of age) were terminated by concussion followed by dislocation. The chest cavity was opened and heparinised solution (10 U/mL) applied around the heart to minimise clotting during dissection. The heart was rapidly excised and placed in warm, oxygenated modified Tyrode solution (PSS, in mM: NaCl 125, NaHCO₃ 25, KCl 5.4, NaH₂PO₄ 1.2, MgCl₂ 1, glucose 5.5, CaCl₂ 1.8, pH to 7.4 with NaOH and oxygenated with 95% O₂/5% CO₂), the ventricular tissue discarded and the sino-atrial node region cleared of any overlying tissue.

Loops of thin suture were tied to the lateral edges of each atrium by directly knotting around a small area of the tissue, taking care to avoid contact with the node itself. One loop was anchored to a hook, which also provided oxygenation to the organ bath, and the other tied to a tension transducer. The preparation was hung in an organ bath filled with PSS, maintained at 37° C. Tension data was digitised using a PowerLabs bridge amplifier and recorded on Chart5 software (all from ADlnstruments, UK). Beating rate was calculated in real-time from the upstroke of the tension signal using the Chart5 Ratemeter function.

Spontaneously-beating atrial preparations were allowed to stabilise for 20 minutes, solution was exchanged for fresh, warmed, PSS and the preparation was again allowed to stabilise for 30 minutes. Drugs were added directly to the organ bath by pipette and allowed to equilibrate for 30 minutes before rate data was sampled.

### Results

As can be seen in FIG. 3, the spontaneous frequency decreases significantly by 11% with 3 μM hydroxychloroquine and by 20% in the presence of 10 μM hydroxychloquine (n=6) when added to the organ bath. A further reduction in spontaneous frequency is observed when 1 μM of a known I_{f} blocker (4-Ethylphenylamino-1,2-dimethyl-6-methylaminopyrimidinium chloride known as ZD7288) is added in the presence of 10 μM of hydroxychloroquine. These experiments demonstrate that the bradycardic activity of the compound of the invention result in a direct effect on the SA node responsible for the cardiac pacemaker activity.

### Example 4

This example studies the chronotropic effects of hydroxychloroquine on anaesthetised adults rats. The method and results are as follows:

### Rat In-Vivo Blood Pressure and Heart Rate Study

Heart rate and arterial blood pressure were measured invasively as a terminal procedure on male Sprague-Dawley rats (300-350 g). General anaesthesia was induced using 5% isoflurane (3 L/min oxygen) in an anaesthetic chamber and then maintained via a facemask using 2% isoflurane with the animal on a pre-heated matt to maintain normal body temperature. The left carotid artery was cannulated with a 3 F portex cannula and connected to a pressure transducer. Data was acquired (200 Hz) in real time using a Biopac M100 system connected to a Dell P4 computer using AcqKnowledge software.

Heart rate was triggered from the arterial blood pressure signal. Intravenous access was gained via the left external jugular vein using a 3 F cannula. After an equilibration period of at least 10 minutes, hydoxychloroquine (dissolved in sterile normal saline for injection and warmed to body temperature) was given as intravenous boluses over 30 seconds (1-30 mg/kg). HCQ was administered in volumes of 50-250 μL and a control experiment showed that equivalent boluses of normal saline did not significantly arterial blood pressure or heart rate on their own. Once a stable response had been reached, measurements were taken as an average over 30 seconds. On completion of the protocol, animals were euthanised with intraperitoneal injection of pentobarbitone.

### Results

As can be seen in FIG. 4, the heart rate decreases significantly without effect on blood pressure at the low doses (1-15 mg, n=6). The percentage transient change in heart rate at 15 mg/kg (the highest dose with no significant change in blood pressure) is 14.3±1.1%.

At very high doses, we observe an effect on blood pressure (which recovers over time). The compound of the invention has a marked reducing effect on heart rate, which is concentration-dependent.

### Example 5

Further experiments (as described in Example 3) were conducted to study the chronotropic effects of hydroxychloroquine on isolated mouse atrial preparation. The frequency of the spontaneous contractile activity is measured using tension transducer techniques. The method and results are as follows:

### Mouse Atrial Preparations

Male CD-1 mice (7-9 weeks of age) were terminated by concussion followed by dislocation.

The chest cavity was opened and heparinised solution (10 U/mL) applied around the heart to minimise clotting during dissection. The heart was rapidly excised and placed in warm, oxygenated modified Tyrode solution (PSS, in mM: NaCl 125, NaHCO₃ 25, KCl 5.4, NaH₂PO₄ 1.2, MgCl₂ 1, glucose 5.5, CaCl₂ 1.8, pH to 7.4 with NaOH and oxygenated with 95% O₂/5% CO₂), the ventricular tissue discarded and the sino-atrial node region cleared of any overlying tissue.

Loops of thin suture were tied to the lateral edges of each atrium by directly knotting around a small area of the tissue, taking care to avoid contact with the node itself. One loop was anchored to a hook, which also provided oxygenation to the organ bath, and the other tied to a tension transducer. The preparation was hung in an organ bath filled with PSS, maintained at 37° C. Tension data was digitised using a PowerLabs bridge amplifier and recorded on Chart5 software (all from ADlnstruments, UK). Beating rate was calculated in real-time from the upstroke of the tension signal using the Chart5 Ratemeter function.

Spontaneously-beating atrial preparations were allowed to stabilise for 20 minutes, solution was exchanged for fresh, warmed, PSS and the preparation was again allowed to stabilise for 30 minutes. Drugs were added directly to the organ bath by pipette and allowed to equilibrate for 30 minutes before rate data was sampled.

### Results

FIG. 5 (A and B) shows the dose-dependent effect of HCQ on spontaneous beating rate in mouse atrial preparations.

FIG. 5(A) is a bar graph which shows the effect of cumulative doses of HCQ on sino-atrial node beating rate in spontaneously beating mouse atrial preparations maintained at 36±1° C. As can be seen in FIG. 5(A) (p<0.05, one-way ANOVA), a 3 μM dose of HCQ elicited a reduction in beating rate of 9±3% from the control. The 10 μM dose of HCQ resulted in a 15±2% reduction in beating rate from control. n=7 for all concentrations. The asterisks in the Fig. are provided to indicate a significant reduction in beating rate when compared to the control.

FIG. 5(B) is a line graph which compares percentage change in atrial beating rate during cumulative HCQ doses with that of time-matched control preparations. FIG. 5(B) shows normalised dose-dependent change in atrial beating rate compared to control preparations. As can be seen, there was no significant effect of time on beating rate in the control preparations (p>0.05, one two-way ANOVA). n=6 for control and n=7 for HCQ. The asterisks in the Fig. indicate a significant effect of the drug.

### Example 6

Further experiments were conducted in isolated guinea pig sinoatrial node cells (as described in Example 2).

This example studies the effects of hydroxychloroquine on ion channels in isolated sinoatrial nodal cells using patch clamp electrophysiological techniques. The method and results are as follows:

### Guinea Pig Sino-Atrial Node Cell Isolation

Male guinea pigs (350-500 g) were sacrificed by concussion followed by cervical dislocation. The heart was rapidly excised and encouraged to beat several times in heparin-containing zero-Ca²⁺ modified Tyrode solution to prevent clotting (10 U/mL). The heart was mounted via the aorta on a constant pressure Langendorff system for retrograde perfusion, with solutions maintained at body temperature. After 2 minutes initial wash with zero Ca ²⁺solution (in mM: NaCl 136, KCl 5.4, NaHCO₃ 12, Na⁺ pyruvate 1, NaH₂PO₄ 1, MgCl₂ 1, EGTA 0.04, glucose 5; gassed with 95% O₂/5% CO₂ to maintain a pH of 7.4), perfusion was switched to re-circulation with collagenase solution (same as zero Ca²⁺ but with 27 mg collagenase/50 mL—type II Worthington, Worthington Biochemical Corp., Lakewood, N.J., USA—0.1 mM CaCl₂ and no EGTA).

Following 25 minutes enzymatic digestion, the right atrium, vena-cavae and pulmonary artery were dissected away from the ventricles. The right atrium was pinned in a Sylgard-coated bath, opened by anterior incision, and the SA node identified from anatomical features and tissue appearance. Under a binocular microscope, the SA node appears as a translucent, smooth, white structure in contrast to the thicker, pinker surrounding atrial tissue. The SA node was dissected into thin strips (˜2×5 mm) and stored in a high potassium medium (in mM: KCl 70, MgCl₂ 5, K⁺ glutamine 5, taurine 20, EGTA 0.04, succinic acid 5, KH₂PO₄ 20, HEPES 5, glucose 10; pH to 7.2 with KOH). Isolation of single SA node myocytes was carried out by gentle trituration.

For all single cell experiments, cells were transferred to a glass coverslip and superfused with oxygenated PSS at 34 ±1° C. using a gravity-fed system running at 2-3 mL/min. HCQ was applied via superfusion, with an electronic switching mechanism resulting in full solution exchange over a period of less than 20 seconds.

### Single Cell Electrophysiology Recordings

Perforated patch clamp recordings were carried out using electrodes pulled from filamented borosilicate glass capillary tubing (GC100 F, Harvard Apparatus Ltd, Kent) using a two-stage vertical puller (Narishige, Japan). Electrode resistances were in the range of 2.8-6.4 MOhm when filled with patch pipette solution containing (in mM): K⁺-aspartate 110, KCl 10, NaCl 5, MgCl₂ 5.2, HEPES 5, K₂ATP 5, pH to 7.2 with KOH. Patch solution also contained amphotericin (250 μg mL⁻¹) to achieve perforation.

Micropipettes were mounted in a Perspex holder containing an Ag/AgCl wire and connected to a CV203BU headstage. All recordings were carried out using an AxoPatch 200B microelectrode system, digitised with a Digidata 1200 A-D converter, and recorded using PCIamp7 software at a sampling rate of 2 kHz (All products in this paragraph, Axon Instruments).

Micropipettes were controlled using an electronic micromanipulator (Burleigh, USA). After positioning close to the cell of interest, the tip potential was manually compensated. Gigaseals were formed by manual suction and the cell left for up to 15 minutes to allow patch perforation and good electrical access using amphotericin. Cells were used in either current clamp mode for the recording of action potentials or voltage clamp mode for the recording of I_{f} currents.

### Action Potential Recordings

Action potentials were recorded from single guinea pig SA node cells under current clamp conditions using the AxoPatch200B microelectrode system and rate measured in Hz using spectral analysis.

### I_{f} Current Recordings

Cell voltage was clamped at −40 mV in the V-clamp configuration. Hyperpolarising pulses of 2 seconds in length were applied at 20 seconds intervals to measure I_{f} current. Current-voltage relations were taken at 0 minutes, 3 minutes and 5 minutes exposure to HCQ by application of successive steps to −80, −100 and −120 mV. Intervening voltage pulses were applied to −100 mV only. Maximum current was measured as the difference between maximum current during the voltage pulse and minimum current at the start of the voltage pulse.

### Results

FIG. 6(A) shows representative traces to show action potentials from the same SAN myocyte before and after superfusion of 1 μM HCQ at 35±2° C. Cells were electrically accessed by the perforated patch method and allowed to fire spontaneous action potentials. Electrodes were used for recording only, with no stimulation of the cells, and action potentials were recorded in the current clamp configuration.

FIG. 6(B) is a bar graph showing absolute action potential firing rate in Hz before and during 1 μM HCQ application (n=6). FIG. 6(B) shows that HCQ reduces action potential firing rate in isolated guinea pig SAN cells. In this regard, AP firing rate was significantly slowed by superfusion of 1 μM HCQ (p<0.05, one-way ANOVA). Compared to control conditions, 1 μM HCQ reduced cellular beating rate by 10±3% after 3 mins of exposure. Cellular beating rate was further reduced by 17±6% at 5 mins of exposure.

The same six cells were further analysed to determine action potential characteristics. As can be seen in FIG. 6(C), 1 μM HCQ significantly reduced the rate of spontaneous diastolic depolarisation (SDD). Further, as can be seen in FIG. 6(D) 1 μM HCQ lengthened the action potential duration. Once again, for both of these analyses, effect of HCQ was significant over time by one-way ANOVA.

The asterisks in FIGS. 6 (B)-(D) are present to indicate a significant difference from the control.

In FIG. 7, (A)-(C) show representative traces of current-voltage relations. (A) shows a representative trace of current-voltage relations under control conditions (in PSS), (B) shows a representative trace of current-voltage relations after 5 mins of exposure to 3 μM HCQ and (C) shows a representative trace of current-voltage relations after 10 mins of wash-out by return to PSS.

FIG. 7(D) shows conductance curves calculated from n=4 cells under the conditions illustrated in FIG. 7(A)-(C), with conductance plotted relative to maximal activation of I(f) under control conditions. Maximal conductance was significantly reduced to 85±6% of control over the course of 5 mins superfusion, whilst voltage of half-activation (V50) and slope of conductance were unchanged.

A lack of effect on V50 and slope are illustrated in FIG. 7(E), where conductance curves are plotted relative to maximal activation of I(f) under control conditions (PSS) and after 5 min of exposure to 3 μM HCQ.

Representative curves to illustrate the effect of HCQ at 1 and 10 μM on voltage steps to −100 mV are shown in FIG. 7(F). Data were collected under control conditions (PSS) and after 5 min of hydroxychloroquine superfusion, during which time voltage steps to −100 mV were repeated at a rate of 1/20 Hz.

FIG. 7(G) shows the average change in I(f) current at the −100 mV step across three concentrations of the drug, measured after 5 min of exposure. There is shown a significant effect of concentration (p<0.05) by ANOVA. n=7 for 1 μM HCQ, n=5 for 3 μM HCQ and n=4 for 10 μM HCQ.

### Example 7

As described in Example 4, Example 7 describes the chronotropic effects of hydroxychloroquine on anaesthetised adults rats. The method and results are as follows:

### Rat In-Vivo Blood Pressure and Heart Rate Study

Heart rate and arterial blood pressure were measured invasively as a terminal procedure on male Sprague-Dawley rats (300-350 g). General anaesthesia was induced using 5% isoflurane (3 L/min oxygen) in an anaesthetic chamber and then maintained via a facemask using 2% isoflurane with the animal on a pre-heated matt to maintain normal body temperature. The left carotid artery was cannulated with a 3 F portex cannula and connected to a pressure transducer. Data was acquired (200 Hz) in real time using a Biopac M100 system connected to a Dell P4 computer using AcqKnowledge software.

Heart rate was triggered from the arterial blood pressure signal. Intravenous access was gained via the left external jugular vein using a 3 F cannula. After an equilibration period of at least 10 minutes, hydoxychloroquine (dissolved in sterile normal saline for injection and warmed to body temperature) was given as intravenous boluses over 30 seconds (1-30 mg/kg). HCQ was administered in volumes of 50-250 μL and a control experiment showed that equivalent boluses of normal saline did not significantly arterial blood pressure or heart rate on their own. Once a stable response had been reached, measurements were taken as an average over 30 seconds. On completion of the protocol, animals were euthanised with intraperitoneal injection of pentobarbitone.

### Results

As can be seen in FIG. 8, during acute in-vivo experiments, heart rate decreases significantly without an effect on blood pressure at low doses of the drug. FIG. 8(A) shows the heart rate responses. There is a significant transient reduction in heart rate observed at cumulative doses of 7.5 mg/kg and above. FIG. 8(B) shows the mean arterial pressure responses. The percentage transient change in heart rate at 15 mg/kg (the highest dose with no significant change in mean arterial blood pressure) is 14.3+/−1.1%. In FIG. 8, the asterisks indicate that p<0.05 baseline versus transient drop (ANOVA/Bonferroni) and the plus signs indicates that p<0.05 baseline versus equilibrated response (ANOVA/Bonferroni).

### Example 8

### Mice in Vivo, Non-Invasive Blood Pressure and Cardiac Contractility Studies

Automated non-invasive tail cuff plethysmography (Visitech 2000, Visitech, USA) was used to determine systolic blood pressure in response to HCQ in drinking water when compared to a control group (n=9/group). C57BL/6 mice were habituated for 5 days and then underwent measurements alternate day for 28 days. All measurements were taken in the morning at the same time of day. Twenty measurements were taken, the first 10 discarded and an average of valid recordings then taken for analysis.

### Results

We assessed safety by performing long term feeding of mice at 100 mg/kg daily for 28 days with HCQ (FIG. 9) and measuring blood pressure by tail cuff plethysmography. As shown in FIG. 9, administration of HCQ (100 mg/kg) in the drinking water did not alter blood pressure from baseline in the treatment group (HCQ baseline 109.7±2.89 mmHg vs end of study 110.7±1.91 mmHg, p=ns, n=9). All animals were weighed daily to ascertain whether they were consuming the drug and no significant weight loss was observed in both control and drug groups (control start weight: 28.08±0.29 g; control end weight: 31.56±0.63 g; HCQ start weight: 27.72±0.36 g; HCQ end weight: 29.99±0.58 g).

### Equivalents

It will readily be apparent that numerous modifications and alterations may be made to the specific embodiments of the invention described above without departing from the principles underlying the invention. All such modifications and alterations are intended to be embraced by this invention.

1. Hydroxychloroquine or a pharmaceutically acceptable salt thereof for use in the prevention and/or treatment of systolic heart failure, diastolic heart failure, sinus tachycardia, cardiac syndrome X and/or essential hypertension.

2. Hydroxychloroquine or a pharmaceutically acceptable salt thereof according to claim 1, wherein the hydroxychloroquine is a racemate.

3. Hydroxychloroquine or a pharmaceutically acceptable salt thereof according to claim 1, wherein the hydroxychloroquine is in the form of an acid-addition salt.

4. Hydroxychloroquine or a pharmaceutically acceptable salt thereof according to claim 1, wherein the salt is a sulfate salt.

5. Hydroxychloroquine or a pharmaceutically acceptable salt thereof according to claim 1, wherein the hydroxychloroquine is to be administered to a human subject at a dosage of up to 1000 milligrams per day.

6. Hydroxychloroquine or a pharmaceutically acceptable salt thereof according to claim 5, wherein the hydroxychloroquine is to be administered to a human subject at a dosage of from 1 to 400 milligrams per day, more preferably from 200 to 400 milligrams per day.

7. Hydroxychloroquine or a pharmaceutically acceptable salt thereof according to claim 1, wherein hydroxychloroquine is administered sequentially, separately or simultaneously with an ACE inhibitor, an angiotensin II receptor blocker, a diuretic, an aldosterone antagonist, calcium channel antagonist, a beta blocker and/or a sinus node If inhibitor.

8. Hydroxychloroquine or a pharmaceutically acceptable salt thereof according to claim 7, wherein hydroxychloroquine is administered sequentially, separately or simultaneously with ivabradine.

9. A combination of hydroxychloroquine or a pharmaceutically acceptable salt thereof and an ACE inhibitor, an angiotensin II receptor blocker, a diuretic, an aldosterone antagonist, a calcium channel antagonist, a beta blocker and/or a sinus node If inhibitor for use in the prevention and/or treatment of systolic heart failure, diastolic heart failure, sinus tachycardia, cardiac syndrome X and/or essential hypertension.

10. A method for preventing and/or treating systolic heart failure, diastolic heart failure, sinus tachycardia, cardiac syndrome X and/or essential hypertension, wherein the method comprises administering to the mammal a therapeutically effective amount of hydroxychloroquine or a pharmaceutical acceptable salt thereof.
